(19) United States
(12) Patent Application Publication
(10) Pub. No.: US 2001/0051779 A1
(43) Pub. Date: Dec. 13, 2001

(54) Medical apparatus

(76) Inventor: Gary Struck, Bensenville, IL (US)

Correspondence Address: Matthew J. Peirce, Esq., 1550 Starlight Canyon Avenue, Las Vegas, NV, 89123, US

(21) Appl. No.: 09/878,068

(22) Filed: Jun. 7, 2001

Related U.S. Application Data
(60) Provisional application No. 60/210,030, filed on Jun. 8, 2000.

(51) Int. Cl. A61F 5/00

(52) U.S. Cl. 602/2; 602/7

(57) ABSTRACT

A new and improved apparatus is disclosed to provide additional comfort for a user when hot or cold temperatures are needed for recreational or medicinal purposes. The present invention would be made from cloth and plastic and would use chemicals to provide hot or cold temperatures as desired by a user. The present invention would be available in different sizes to accommodate different body areas such as the wrist, waist, and head. The exact chemical mix would vary, depending on whether the present invention would be used for medical or recreational purposes.

### I. BACKGROUND OF THE INVENTION

The present invention concerns that of a new and improved apparatus used to provide additional comfort for a user when hot or cold temperatures are needed for recreational or medicinal purposes.

### II. DESCRIPTION OF THE PRIOR ART

U.S. Pat. No. 5,787,505, issued to Piwko et al., discloses one or more pouches in combination with a shirt which receives a cooling and/or warming pack.

U.S. Pat. No. 5,000,176, issued to Daniel, discloses a therapeutic wrap for holding a heat transference device, such as an ice pack or a hot pack, in contact with the human body.

U.S. Pat. No. 4,776,042, issued to Hanson et al., discloses a cryokenetic headband having a closable pocket for retaining a cold pack material.

### III. SUMMARY OF THE INVENTION

The present invention concerns that of a new and improved apparatus used to provide additional comfort for a user when hot or cold temperatures are needed for recreational or medicinal purposes. The present invention would be made from cloth and plastic and would use chemicals to provide hot or cold temperatures as desired by a user. The present invention would be available in different sizes to accommodate different body areas such as the wrist, waist, and head. The exact chemical mix would vary, depending on whether the present invention would be used for medical or recreational purposes.

There has thus been outlined, rather broadly, the more important features of a health aid apparatus in order that the detailed description thereof that follows may be better understood and in order that the present contribution to the art may be better appreciated. There are, of course, additional features of the health aid apparatus that will be described hereinafter and which will form the subject matter of the claims appended hereto.

In this respect, before explaining at least one embodiment of the health aid apparatus in detail, it is to be understood that the health aid apparatus is not limited in its application to the details of construction and to the arrangements of the components set forth in the following description or illustrated in the drawings. The health aid apparatus is capable of other embodiments and being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present health aid apparatus. It is important, therefore, that the claims be regarded as including such equivalent constructions insofar as they do not depart from the spirit and scope of the present invention.

It is therefore an object of the present invention to provide a health aid apparatus which has all of the advantages of the prior art and none of the disadvantages.

It is another object of the present invention to provide a health aid apparatus which may be easily and efficiently manufactured and marketed.

It is another object of the present invention to provide a health aid apparatus which is of durable and reliable construction.

It is yet another object of the present invention to provide a health aid apparatus which is economically affordable and available for relevant purchasing government entities.

Other objects, features and advantages of the present invention will become more readily apparent from the following detailed description of the preferred embodiment when considered with the attached drawings and appended claims.

### IV. BRIEF DESCRIPTION OF THE DRAWINGS

FIG. 1 shows a perspective view of the present invention.

FIG. 2 shows a top view of the present invention.

FIG. 3 shows the present invention in use by a user.

### V. DESCRIPTION OF THE PREFERRED EMBODIMENT

FIG. 1 shows a perspective view of the present invention. Band 2 can be seen with hollow compartment 4 located inside of band 2. Band 2 is a continuous loop which would be preferably be at least 1 inch in height and would include top border 6 and bottom border 8. The height of band 2 would be different, depending on whether band 2 would be intended for a male user or a female user. Band 2 would preferably be fabricated of terry cloth material. Band 2 would have a one to two inch length of elastic 10 which would allow band 2 to be used in a variety of sizes of wrists, waists, or heads.

Band 2 would be available in one of several sizes. Band 2 would be made available in various sizes, each of which would be specifically designed for a person's wrist, waist, or head. The presence of elastic 10 on each band 2 would allow a particular band 2 to fit almost any person with respect to the intended function of that particular band.

Band 2 would be made available in a wide variety of colors, such as yellow, tan, red, blue, green, or other commonly used colors. In addition, band 2 could be designed in seasonal themes, such as 4^{th} of July, Christmastime, Easter, and other possible themes.

Band 2 would come with inner compartment 12, which would be located in hollow compartment 4. Chemical mixture 14 would be located within hollow compartment 4, while chemical mixture 16 would be located within inner compartment 12. Inner compartment 12 would be easily breakable by a user, while at the same time, band 2 would be strong enough to not be easily punctured or broken.

If band 2 was designed to be a “hot band,” then the mixing of chemical mixture 14 and chemical mixture 16 would produce heat for a temporary amount of time. A user would accomplish this task by grasping inner compartment 12 through band 2 and breaking inner compartment 12, which would cause chemical mixture 14 and chemical mixture 16 to mix. The mixing of chemical mixture 14 and chemical mixture 16 would produce heat. Once this would happen, a user could then wrap band 2 around the afflicted body portion or part in an effort to provide heat to the injured or afflicted area. After use, band 2 would be discarded.

If band 2 was designed to be to produce cold temperatures, then the mixing of chemical mixture 14 and chemical mixture 16 would produce cold temperatures for a temporary amount of time. A user would accomplish this task by grasping inner compartment 12 through band 2 and breaking inner compartment 12, which would cause chemical mixture 14 and chemical mixture 16 to mix. The mixing of chemical mixture 14 and chemical mixture 16 would produce coldness. Once this would happen, a user could then wrap band 2 around the afflicted body portion or part in an effort to provide coldness to the injured or afflicted area. After use, band 2 would be discarded.

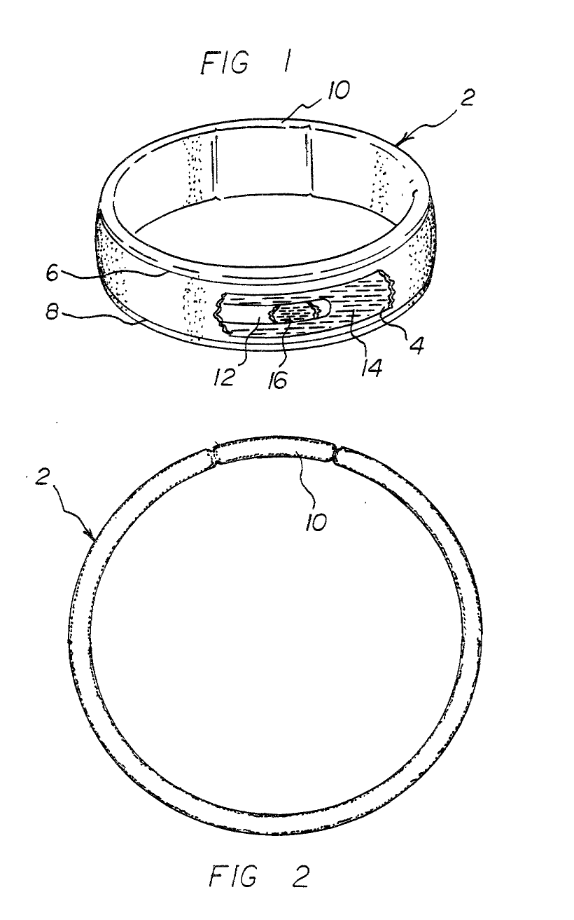

FIG. 2 shows a top view of band 2 of the present invention. As can be seen, band 2 includes elastic 10.

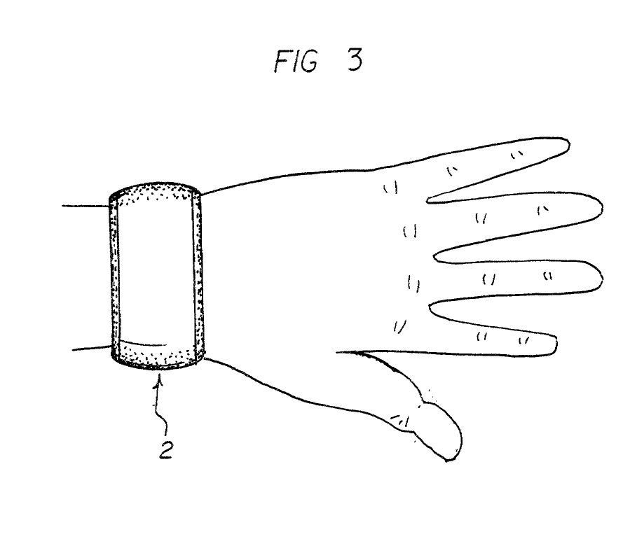

FIG. 3 shows the present invention in use by a user. As can be seen, band 2 is being used by a user to alleviate an affliction and/or injury to a troubled body area.

What I claim as my invention is:

1. A medical apparatus for producing heat comprising:
(a) a continuous band preferably fabricated from a material suitable for contact with the human body, the continuous band including a length of elastic approximately one to two inches in length, the continuous band preferably being at least one inch in height,
(b) a hollow compartment located within the continuous band, the hollow compartment containing a first chemical mixture, the hollow compartment being self-contained and tubular in nature, the hollow compartment being fabricated from a flexible material, the first chemical mixture preferably being in liquid form
(c) an inner compartment located within the hollow compartment, the inner compartment containing a second chemical mixture, the inner compartment being self-contained and tubular in nature, the inner compartment being fabricated from an easily breakable material,
(d) wherein a user would break the inner compartment in one or more locations by handling the medical apparatus, causing the first chemical mixture and the second chemical mixture to mix within the hollow compartment, wherein the mixing of the first chemical mixture and the second chemical mixture will cause a chemical reaction to occur that generates heat, the chemical reaction designed to generate heat for a specified period of time, wherein a user would place the medical apparatus around a body part for medical treatment.

2. A medical apparatus according to claim 1 wherein the continuous band would be fabricated from terry cloth material.

3. A medical apparatus according to claim 1 wherein the hollow compartment would be located within the continuous band but would not be located within the length of elastic.

4. A medical apparatus for producing cold temperatures comprising:
(a) a continuous band preferably fabricated from a material suitable for contact with the human body, the continuous band including a length of elastic approximately one to two inches in length, the continuous band preferably being at least one inch in height,
(b) a hollow compartment located within the continuous band, the hollow compartment containing a first chemical mixture, the hollow compartment being self-contained and tubular in nature, the hollow compartment being fabricated from a flexible material, the first chemical mixture preferably being in liquid form,
(c) an inner compartment located within the hollow compartment, the inner compartment containing a second chemical mixture, the inner compartment being self-contained and tubular in nature, the inner compartment being fabricated from an easily breakable material,
(d) wherein a user would break the inner compartment in one or more locations by handling the medical apparatus, causing the first chemical mixture and the second chemical mixture to mix within the hollow compartment, wherein the mixing of the first chemical mixture and the second chemical mixture will cause a chemical reaction to occur that generates cold temperatures, the chemical reaction designed to generate cold temperatures for a specified period of time, wherein a user would place the medical apparatus around a body part for medical treatment.

5. A medical apparatus according to claim 4 wherein the continuous band would be fabricated from terry cloth material.

6. A medical apparatus according to claim 4 wherein the hollow compartment would be located within the continuous band but would not be located within the length of elastic.
